(19) United States
(12) Patent Application Publication
(10) Pub. No.: US 2024/0042083 A1
(43) Pub. Date: Feb. 8, 2024

(54) DIFFUSER

(71) Applicant: Rimports, LLC, Prove, UT (US)

(72) Inventors: Alan K. Farrell, Salt Lake City, UT (US); Clark Kalmar, Saratoga Springs, UT (US); Jeff Goodsell, Springville, UT (US)

(21) Appl. No.: 18/230,597

(22) Filed: Aug. 4, 2023

Related U.S. Application Data
(60) Provisional application No. 63/395,300, filed on Aug. 4, 2022.

(51) Int. Cl. A61L 9/03

(52) U.S. Cl. CPC A61L 9/03; H01R 13/52

(57) ABSTRACT

A plug-in diffuser, such as a fragrance diffuser, has a configuration that preserves a substance to be diffused, such as a fragrance. A configuration of a body of the plug-in diffuser may prevent the substance to be diffused from contacting an electrical plug of the fragrance diffuser and/or from entering into an electrical outlet. A receptacle in a top of the body of the plug-in diffuser may receive a reservoir of the substance to be diffused. A canopy assembled with the body may provide decorative effects (e.g., appearance, lighting, etc.).

### CROSS-REFERENCE TO RELATED APPLICATION

A claim for priority is made to the Aug. 4, 2022 filing date of U.S. Provisional Patent Application No. 63/395,300, titled DIFFUSER (“the '300 Provisional Application”). The entire disclosure of the '300 Provisional Application is hereby incorporated herein.

### TECHNICAL FIELD

This disclosure relates generally to plug-in diffusers, including, but not limited to, fragrance diffusers. More specifically, a diffuser according to this disclosure may have a configuration that preserves a substance that is to be diffused, such as a fragrance. A diffuser according to this disclosure may include a body with a configuration that prevents a substance that is to be diffused from contacting an electrical plug of the fragrance diffuser and/or from entering into an electrical outlet. A diffuser according to this disclosure may include a body with a receptacle that can receive a reservoir of substance that is to be diffused in a top of the body; such a diffuser may include a canopy that may provide decorative effects (e.g., appearance, lighting, etc.).

### SUMMARY

In various embodiments, a diffuser according to this disclosure may comprise a so-called plug-in diffuser, which can operate when plugged into an electrical outlet. Such a diffuser includes a body, a receptacle, a converter, a diffusion nozzle, a power supply, and an electrical plug. The diffuser may also include a plug protrusion.

The body may include a front, sides, a back, a top, a bottom, an exterior, and an interior. The body may define or carry other elements of the diffuser. For example, the receptacle may be defined within the top of the body. The converter may be carried within an interior of the body. The diffusion nozzle may extend from the interior of the body to and optionally through an opening in the front of the body. The plug protrusion may comprise a protruding portion of the back of the body. The electrical plug may protrude from the plug protrusion.

The receptacle, which receives a reservoir (e.g., a reservoir for a substance to be diffused, such as a fragrance, another liquid, etc.), communicates with the interior of the body. In some embodiments, the receptacle may be located in the top of the body. The receptacle may include a coupler, which enables it to receive and retain an opening of the reservoir. The receptacle (e.g., the coupler thereof, etc.) may also have a configuration that provides for the communication of the substance (e.g., a fragrance, another liquid, etc.) carried by the reservoir to the converter.

The converter may be located within an interior of the body, where it can receive the substance from the receptacle (e.g., through the coupler, etc.; from a reservoir received by the receptacle, etc.). When the diffuser receives power (e.g., from a power supply associated with the electrical plug, etc.), it may be turned on and facilitate diffusion of the substance it receives from the receptacle. Stated another way, the converter may place the substance in a diffusible state (e.g., a vapor, atomized droplets, etc.). In some embodiments, the converter may comprise a heater or other apparatus that can change a state of the substance (e.g., from a liquid to a gas, etc.). In other embodiments, the converter may comprise a pump or other apparatus that conveys the substance under pressure to facilitating atomization of the substance. The converter may facilitate communication of the substance to the diffusion nozzle.

The diffusion nozzle receives the substance from the converter and directs the substance through an opening in the front of the body of the diffuser to a location outside of the diffuser. Additionally, the diffusion nozzle may atomize or otherwise facilitate dispersion of the substance through the air outside of the diffuser. The diffusion nozzle may be oriented in such a way as to recapture any substance that is not dispersed into the air around the diffuser. Without limitation, the diffusion nozzle may be oriented at an angle (e.g., a somewhat upwardly oriented, or diagonally oriented, angle of less than 90° to vertical; etc.) that facilitates collection of an undiffused portion of the substance to be diffused and enables the undiffused portion of the substance to flow back to the converter and/or the receptacle.

The power supply may comprise any suitable power supply. The power supply provides power to electronics and electrically operated components of the diffuser.

In some embodiments, the diffuser may include a light source carried by the body. The light source may provide one or more lighting effects, as described in further detail hereinafter.

A diffuser may include one or more controls. Without limitation, the diffuser may include a power switch (i.e., an on/off switch) (e.g., a push-button switch, etc.), which may control the flow of power from a power supply (e.g., through an electrical outlet, etc.) to the converter and any other electrical components of the diffuser. As another example, the diffuser may include one or more controls that are used to control the amount of a substance the diffuser diffuses. The diffuser may also include other types of controls (e.g., lighting controls, sound controls, controls that enable operation of the diffuser to be wirelessly controlled (e.g., by way of a remote control, a smart phone, a tablet computer, etc.), etc.).

A shape of the exterior of the body, including a shape of the plug protrusion, may prevent any liquid (e.g., the substance to be diffused, etc.) that collects on the exterior of the body from flowing to the electrical plug. The shape of the exterior of the body, including the shape of the plug protrusion, may prevent any liquid that collects on the exterior of the body from flowing into an electrical socket with which the electrical plug has been coupled.

The electrical plug may include a plurality of prongs that extend from the plug protrusion to facilitate coupling of the diffuser to a power source (e.g., an electrical outlet, or wall socket, etc.). In some embodiments, the electrical plug may be positionable in (e.g., rotated to, etc.) a plurality of different orientations; such an electrical plug may enable the diffuser to be positioned upright or substantially upright regardless of the orientation of an electrical outlet (e.g., upright, sideways, upside down, diagonal, etc.) the electrical plug is coupled with, or plugged into. When coupled with a power source (e.g., an electrical outlet, etc.), the electrical plug may facilitate the communication of power from the power source to the converter, which may facilitate operation of the diffuser. In addition, the electrical plug may facilitate the communication of power from the power source to a light source of the diffuser, as well as to any other electrically operated components of the diffuser.

The diffuser may also include a canopy. The canopy may be removably positionable over the receptacle and, thus, over a reservoir that has been received by (e.g., coupled to, etc.) the receptacle. The canopy may have a decorative exterior. The canopy may include an exterior wall that receives light from a light source of the diffuser (e.g., a light source that directs light to a canopy positioned over the receptacle, etc.). The exterior wall of the canopy may transmit light in a manner that illuminates the canopy (e.g., illuminates the exterior wall of the canopy, etc.). Such a canopy may also transmit light to other features of the diffuser, such as decorative charms that may be assembled with the canopy, which may enable illumination of the other features.

In another aspect, methods of using a diffuser are disclosed. For example, methods for recycling a diffused substance are disclosed. A method of recycling a diffused substance (e.g., a fragrance, etc.) with a dispenser may include directing a diffused substance that collects on an exterior of the dispenser (e.g., by condensation, etc.) at a location near a nozzle of the dispenser back into the dispenser (e.g., to a converter that converts the substance to a diffusable state, to a reservoir for the substance, etc.). Other methods include, but are not limited to, preventing a diffused substance from collecting on an electrical plug of the diffuser, and providing lighting effects on the diffuser.

Other aspects of the disclosed subject matter, as well as features and advantages of various aspects of the disclosed subject matter, should be apparent to those of ordinary skill in the art through consideration of the disclosure, the accompanying drawings, and the appended claims.

### BRIEF DESCRIPTION OF THE DRAWINGS

In the drawings:

FIG. 1 is a front perspective view of an embodiment of a diffuser according to this disclosure;

FIG. 2 is a side view of the embodiment of the diffuser shown in FIG. 1;

FIG. 3 is a second side view of the embodiment of the diffuser shown in FIG. 1;

FIG. 4 is a rear view of the embodiment of the diffuser shown in FIG. 1, depicting an electrical plug of the diffuser in a first orientation;

FIG. 4A is a rear view of the embodiment of the diffuser shown in FIG. 1, depicting the electrical plug of the diffuser in a second orientation, which differs from the first orientation;

FIG. 5 is a side perspective view of the embodiment of the diffuser shown in FIG. 1, illustrating a power switch on a side of the body of the diffuser;

FIG. 6 is a rear side perspective of the embodiment of the diffuser shown in FIG. 1;

FIG. 7 is a perspective view of the embodiment of the diffuser shown in FIG. 1, with the electrical plug of the diffuser coupled with, or plugged into, an electrical outlet;

FIG. 8 is an exploded perspective view of the embodiment of the diffuser shown in FIG. 1; and

FIG. 9 is a cross-sectional representation of the embodiment of the diffuser shown in FIG. 1.

### DETAILED DESCRIPTION

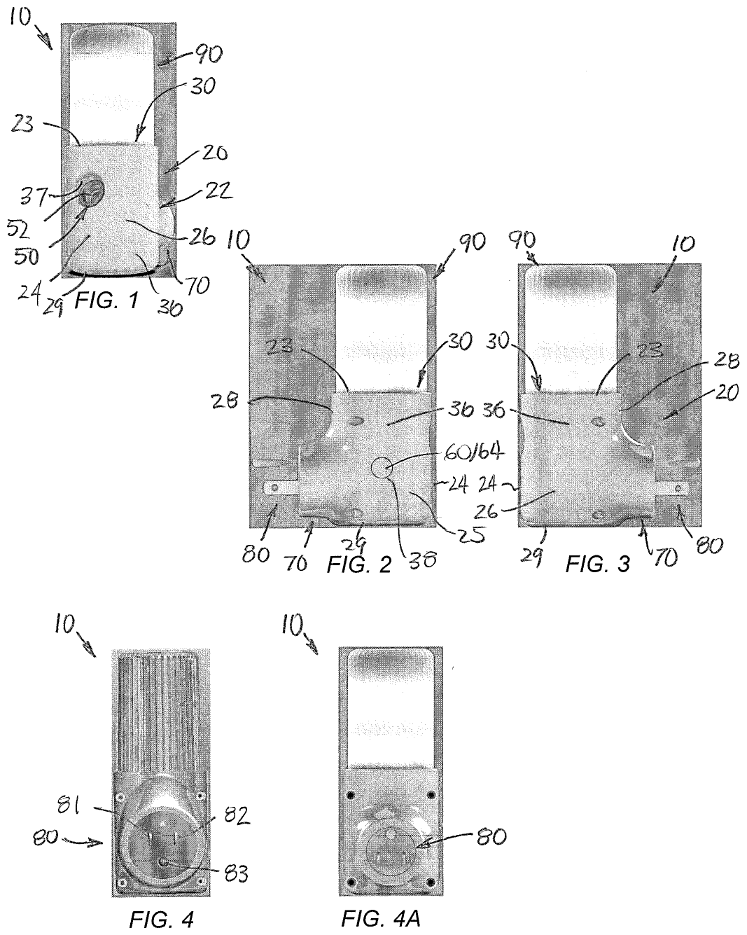

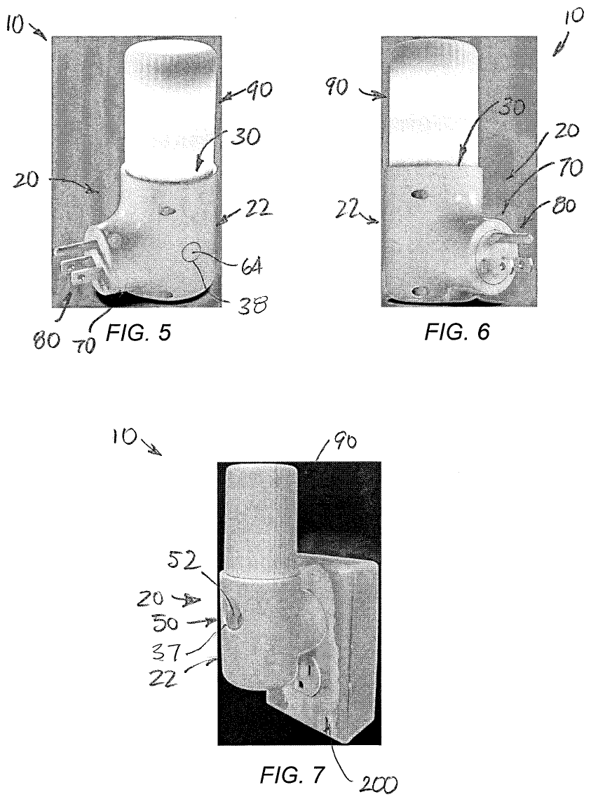

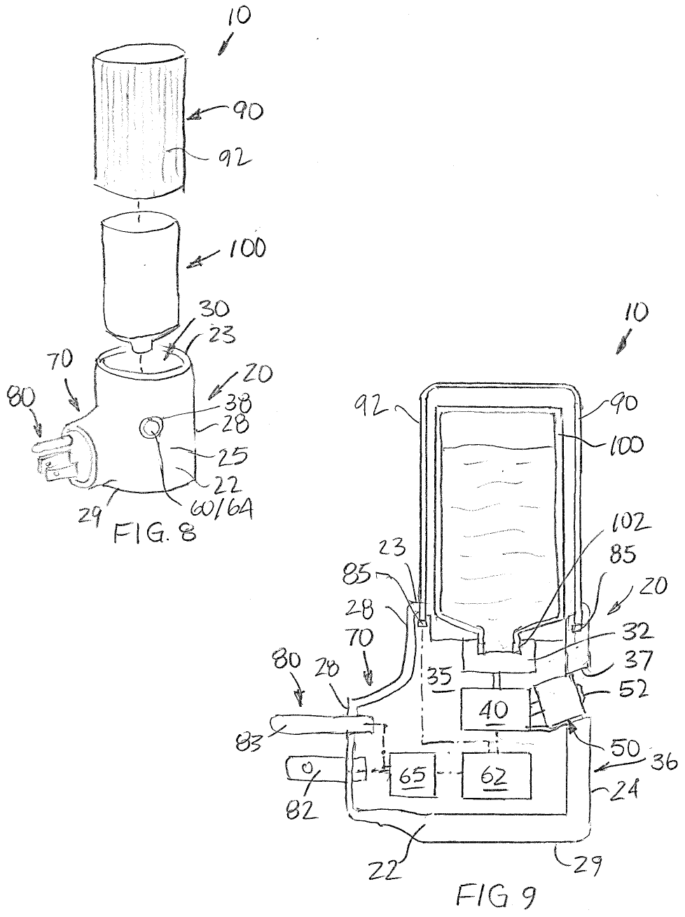

FIGS. 1-9 depict an embodiment of a diffuser 10. The diffuser 10 includes a body 20, a receptacle 30, a converter 40 (FIG. 9), a diffusion nozzle 50 (FIGS. 1 and 9), one or more controls 60 (FIGS. 2 and 8), a power supply 65 (FIG. 9), and an electrical plug 80. The diffuser 10 may also include a plug protrusion 70. The diffuser 10 is configured to receive a receptacle 100, which carries a substance to be diffused (e.g., a fragrance, etc.). Other optional components of the diffuser 10 include a canopy 90 positionable atop the body 20 and lighting effects 95 carried by the body 20.

The body 20 of the diffuser 10 comprises a housing 22. As shown in FIGS. 1-3, 8, and 9, the housing 22 of the body 20 may include a top 23, a bottom 29, a front 24, a back 28, and sides 25 (FIGS. 2 and 8) and 26 (FIGS. 1 and 3). As best seen in FIGS. 8 and 9, the receptacle 30 may be defined by or within the top 23 of the housing 22. As best seen in FIG. 9, the housing 22 may also define an interior 35 and an exterior 36. One or more openings 37 (FIGS. 1 and 9), 38 (FIGS. 2 and 8), etc., may be formed through the housing 22. A front opening 37 (FIGS. 1 and 9) may be formed through the front 24 of the housing 22. A side opening 38 (FIGS. 2 and 8) may be formed through a side 25, 26 of the housing 22.

The housing 22 may carry, or at least partially carry, other components of the diffuser 10. For example, as shown in FIG. 9, the converter 40, the diffusion nozzle 50, and the power supply 65 may be carried at least partially within the interior 35 of the housing 22. The control(s) 60 (e.g., the power switch 64) (FIGS. 2 and 8) and the electrical plug 80 may be accessible from the exterior 36 of the housing 22.

As shown in FIGS. 8 and 9, the receptacle 30 has a configuration that enables it to receive the reservoir 100. With reference to FIG. 9, the receptacle 30 may include a coupler 32 (e.g., helical threading, a snap-fit coupler, etc.) (FIG. 9) that enables it to receive and retain an opening 102 of the reservoir 100. The receptacle 30 (e.g., the coupler 32 thereof, etc.) may also have a configuration (e.g., one or more conduits, one or more valves, etc., as known in the art) that provides for the controlled communication of the substance carried by the reservoir 100 to the converter 40.

With continuing reference to FIG. 9, the converter 40 may be carried within the interior 35 of the housing 22. The converter 40 receives the substance from the receptacle 30 (e.g., through the coupler 32, conduit(s), valve(s), etc.; from a reservoir 100 received by the receptacle 30, etc.). The converter 40 may comprise a heater, vaporizer, or other apparatus that can change a state of the substance (e.g., from a liquid to a gas, etc.). Alternatively, the converter 40 may comprise a pump or other apparatus that conveys the substance under pressure and atomizes the substance. The converter 40 may facilitate communication of the substance to the diffusion nozzle (e.g., by way of one or more conduits, one or more valves, etc.).

With added reference to FIG. 1, the diffusion nozzle 50 may also be carried within the interior 24 of the housing 22. The diffusion nozzle 50 may be positioned to receive the substance from the converter 40. The diffusion nozzle 50 may facilitate dispersion of the substance into the air outside of the diffuser 10. For example, a gaseous or vaporized substance may flow from an emission face 52 of the diffusion nozzle 50. As another example, the diffusion nozzle 50 may atomize or further atomize a substance flowing therethrough.

An emission face 52 of the diffusion nozzle 50 may be exposed through the front opening 37 through the housing 22 to enable the substance to be emitted, or diffused, to the air around the diffuser 10. In some embodiments, the front opening 37 may receive the emission face 52. The emission face 52 may be recessed relative to the exterior 36 of the housing 22.

The diffusion nozzle 50 and its emission face 52 and, optionally, the front opening 37 through the housing 22 may be oriented in such a way as to recapture any substance that is not dispersed into the air around the diffuser 10. In some embodiments, the diffusion nozzle 40 and/or a lower surface of the front opening 37 may be oriented (e.g., at a downward slope, or angle, etc.) to allow any of the diffused substance that collects (e.g., condenses, etc.) within the front opening 37 or on the emission face 52 of the diffusion nozzle 50 to flow (e.g., under force of gravity, etc.) toward the interior 35 of the housing 22. Without limitation, the diffusion nozzle 50 and/or the lower surface of the front opening 37 may be oriented at an angle (e.g., a somewhat upwardly oriented, or diagonally oriented, angle of less than 90° to vertical; etc.) that facilitates collection of an undiffused portion of the substance. The collected substance may be returned, or recycled, (e.g., by way of conduits, etc.) to the converter 40 (as shown) and/or the receptacle 30.

The controls 60 of the diffuser 10 may also be carried by the housing 22. The controls 60 may include electronics 62, as represented in FIG. 9, that enable and control operation of the diffuser 10, including its primary function (i.e., diffusion) and any secondary functions (e.g., lighting effects, sound effects, wireless connectivity to an external control device (e.g., a remote control, a smart phone, a tablet computer, etc.), etc.), etc.). As depicted by FIGS. 2 and 8, the controls 60 may include a power switch 64 (e.g., a push button switch, etc.), which is accessible through the side opening 38 in the housing 22. The power switch 64 may control the flow of power from the power supply 65 (FIG. 9) of the diffuser 10 to the converter 40 (FIG. 9) and any other electrical components of the diffuser 10. In some embodiments, the power switch 64 may be rotated to provide control over the extent to which the diffuser 10 (e.g., its converter 40, etc.) diffuses a substance (i.e., an amount of the substance (e.g., volume, etc.) diffused over a fixed period of time (e.g., per second, per minute, per hour, etc.)).

Referring again to FIG. 9, the power supply 65 may comprise any suitable power supply. The power supply 65 provides electric current power to electronics and electrically operated components of the diffuser 10. Without limitation, the power supply 65 may comprise an electric circuit that changes the type of electric current supplied to electronics and electrically operated components of the diffuser 10. For example, the power supply 65 may convert AC (alternating current) received through the electrical plug 80 the diffuser 10 to DC (direct current) that enables any electronics 62 and other electrically operated components (e.g., the converter 40, any lights, etc.) of the diffuser 10 to operate (i.e., the power supply 65 may comprise a rectifier or the like). In some embodiments, the power supply 65 may store power (e.g., it may comprise a battery, etc.).

As shown in FIGS. 4, 7, and 9, the electrical plug 80 (FIGS. 4 and 9) of the diffuser 10 provides power (e.g., electricity) from an external power source 200 (FIG. 7) (e.g., an electrical outlet 200, etc.) to the power supply 68 of the diffuser 10. The electrical plug 80 may include a plurality of prongs 81, 82, 83 (FIGS. 4 and 7) that facilitate coupling of the diffuser to the power source 200 (e.g., the electrical outlet, etc.). The electrical plug 80 may have any suitable configuration (e.g., it may be a NEMA (National Electrical Manufacturers Association) 1-15 ungrounded (Type A) plug, a NEMA 5-15 grounded (Type B) plug, etc.). The electrical plug 80 protrudes from the back 28 of the housing 22 of the body 20 of the diffuser 10. In some embodiments, the electrical plug 80 may be located adjacent to the bottom 29 of the housing 22.

In some embodiments, the electrical plug 80 may be positionable in (e.g., rotated to, etc.) a plurality of different orientations. FIG. 4 shows the electrical plug 80 in an upright orientation while FIG. 4A shows the electrical plug 80 in an inverted orientation. The electrical plug 80 may also be moved to other orientations (sideways, diagonal, etc.). Such an electrical plug 80 may enable the diffuser 10 to be positioned upright or substantially upright regardless of the orientation of an electrical socket (e.g., upright, sideways, upside-down, diagonal, etc.) the electrical plug 80 is coupled with, or plugged into, to couple the diffuser 10 to the external power source 200 (FIG. 7).

In some embodiments, such as that depicted by FIGS. 1-9, the electrical plug 80 protrudes from a plug protrusion 70 that, in turn, protrudes from the housing 22 of the body 20 of the diffuser 10. The plug protrusion 70 protrudes from the back 28 of the housing 22 and may be located adjacent to the bottom 29 of the housing 22.

A shape of the exterior of the body 20, including a shape and a length of any plug protrusion 70, may prevent any liquid (e.g., the substance to be diffused, etc.) that collects on the exterior 36 of the housing 22 of the body 20 from flowing to the electrical plug 80 and into an electrical socket with which the electrical plug 80 has been coupled. Without limitation, a surface of the exterior 36 of the housing 22 and an exterior surface 76 of the plug protrusion 70 may have shapes (e.g., curves, contours, channels, etc.) that prevent fluid from flowing into the electrical plug 80 and instead direct the fluid to a bottom 79 of the plug protrusion 70 and or the bottom 29 of the housing 22.

As shown in FIG. 9, the diffuser 10 may include a light source 85 carried by the body 20. The light source 85 may comprise one or more LEDs (light-emitting diodes). The light source 85 may operate under control of electronics 62 of the diffuser 10, which may enable the light source 85 to provide one or more lighting effects (e.g., to emit one or more colors, to emit different colors in succession, etc.).

As FIGS. 8 and 9 illustrate, the canopy 90 of the diffuser 10 may be removably positionable over the receptacle 30 of the body 20 of the diffuser and, thus, over a reservoir 100 that has been received by (e.g., coupled to, etc.) the receptacle 30. The canopy 90 may have a decorative exterior. The canopy 90 may include an exterior wall 92 that receives light from a light source 85 of the diffuser 10 (e.g., a light source 85 oriented to direct light to the canopy 90, when positioned over the receptacle 30, etc.). The exterior wall 92 of the canopy 90 may transmit light in a manner that illuminates the canopy 90 (e.g., illuminates the exterior wall 92 of the canopy, etc.). Such a canopy 90 may also transmit light to other features of the diffuser, such as decorative charms that may be assembled with the canopy 90, which may enable illumination of such other features.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A plug-in fragrance diffuser, comprising:
a body including a front, a back, a top, a bottom, an exterior, and an interior;
a plug protrusion extending from the back of the body;
an electrical plug for connecting the plug-in fragrance diffuser to an electrical socket, including a plurality of prongs extending from the plug protrusion;
a receptacle in the top of the body that receives a reservoir;
a diffuser within the interior of the body and in electrical communication with the electrical plug and fluid communication with the receptacle; and
a diffusion nozzle in fluid communication with the diffuser and oriented to diffuse a diffused substance from the front of the body.

2. The plug-in fragrance diffuser of claim 1, wherein shapes of the exterior of the body and the plug protrusion prevent liquid on the exterior of the body from flowing to the electrical plug.

3. The plug-in fragrance diffuser of claim 2, wherein the shapes of the exterior of the body and the plug protrusion prevent the liquid on the exterior of the body from flowing into the electrical socket.

4. The plug-in fragrance diffuser of claim 1, wherein the diffusion nozzle is oriented at an angle that facilitates collection of an undiffused portion of the diffused substance and enables the undiffused portion of the diffused substance to flow to the diffuser and/or the receptacle.

5. The plug-in fragrance diffuser of claim 1, further comprising:
a canopy removably positionable over the receptacle.

6. The plug-in fragrance diffuser of claim 5, wherein the canopy carries the reservoir.

7. The plug-in fragrance diffuser of claim 5, wherein the canopy has a decorative exterior.

8. The plug-in fragrance diffuser of claim 5, further comprising:
a light source that directs light to the canopy,
the canopy including an exterior wall that receives light from the light source and transmits the light in a manner that illuminates the canopy.

9. The plug-in fragrance diffuser of claim 8, further comprising:
a charm connectable to the canopy in such a way that the charm receives light from the canopy and transmits the light to at least partially illuminate the charm.

10. A plug-in fragrance diffuser, comprising:
a body including a front, a back, a top, a bottom, an exterior, and an interior;
an electrical plug for connecting the plug-in fragrance diffuser to an electrical socket, including a plurality of prongs extending from the back of the body;
a receptacle that receives a reservoir;
a diffuser within the interior of the body and in electrical communication with the electrical plug and fluid communication with the receptacle; and
a diffusion nozzle in fluid communication with the diffuser and oriented to diffuse a diffused substance from the front of the body at an angle that facilitates collection of an undiffused portion of the diffused substance and enables the undiffused portion of the diffused substance to flow to the diffuser and/or the receptacle.

11. The plug-in fragrance diffuser of claim 10, wherein the receptacle is associated with the top of the body.

12. The plug-in fragrance diffuser of claim 11, further comprising:
a canopy removably positionable over the receptacle.

13. The plug-in fragrance diffuser of claim 12, wherein the canopy has a decorative exterior.

14. The plug-in fragrance diffuser of claim 12, further comprising:
a light source that directs light to the canopy,
the canopy including an exterior wall that receives light from the light source and transmits the light in a manner that illuminates the canopy.

15. The plug-in fragrance diffuser of claim 14, further comprising:
a charm connectable to the canopy in such a way that the charm receives light from the canopy and transmits the light to at least partially illuminate the charm.

16. The plug-in fragrance diffuser of claim 12, wherein the canopy carries the reservoir.

17. A method of recycling fragrance with a fragrance dispenser, comprising directing fragrance that collects on an exterior of the fragrance dispenser at a location near a nozzle of the fragrance dispenser back into the fragrance dispenser.

18. The method of claim 17, wherein directing the fragrance that collects comprises directing fragrance that condenses on the exterior of the fragrance dispenser.

19. The method of claim 17, wherein directing the fragrance that collects comprises directing the fragrance that collects back to a converter that converts the fragrance to a diffusible state.

20. The method of claim 17, wherein directing the fragrance that collects comprises directing the fragrance that collects to a reservoir for the fragrance.
